SEQUENCE LISTING (15 sequences)

SEQ ID NO: 1 - DNA, 1716 bp, Rhodosporidium toruloides
atgctccttaacctcttcaccctcgcctccctcgctgcgacgctccagctcgcctttgcctctccgacctccctcgtccgccgcacgaacccaaacgagccccctcccgtcgtcgacctcggctacgcccgctaccaaggctacttgaacgagaccgccggactctactggtggcgcggaatccgctacgcctcggctcagcgcttccaggctcctcagacgcccgcgacgcacaaggccgtccgcaacgcgactgagtatggaccgatctgttggccggctagcgagggaaccaacacgaccaagggcttgccgccgcctagcaacagctcgagcagcgcgccgcagaaacaggcgtcggaggattgcctcttcctcaatgtcgttgcccccgccggctcgtgcgagggcgacaatcttcccgtcctcgtctacattcacggaggtggctacgccttcggcgatgcgagcaccggcagcgactttgccgccttcaccaagcacacgggaaccaagatggtcgttgtaaatctccagtaccgtctcggcagctttggtttcctcgctggccaagccatgaaggactacggtgtaacgaacgccggcttgcttgaccagcaattcgcccttcaatgggttcaacagcacgtctcgaagttcggcggcaaccccgatcacgttacgatttggggcgagtctgcaggcgcagggtccgttatgaaccagatcattgcgaacggcggcaacaccgtcaaggctctcggtctcaagaagcccctcttccacgctgccatcggctcctccgtcttcctcccctaccaagccaagtacaactcccccttcgccgagctgctctactcccaactcgtctcggcgacaaactgcaccaaagccgcctcgtccttcgcttgcctcgaagctgtcgacgctgcggcgctcgctgcggcgggcgtgaagaactcggcggcgttcccgttcgggttttggtcgtatgtcccggtcgtcgacgggaccttcttgactgagcgcgcgtcgctccttctcgccaagggcaagaagaacctcaatggcaacctcttcaccgggatcaacaacctcgacgaaggattcatattcactgacgccactattcagaacgacacgatcagcgaccagtcgcagcgcgtctcccagttcgaccgcctcctcgccggcctcttcccctacatcacctcggaggagcgccaggccgtcgcgaagcagtacccgatctccgacgcgccgtcaaagggcaacaccttctctcgcatctcggccgtcatcgcggactcgaccttcgtctgcccgacctactggaccgccgaggcgttcggctcgtccgcccacaagggcctcttcgactacgcgccggctcaccacgcgaccgacaactcgtactacatcggctccatctggaacggcaagaagtcggtctcgtccgtccagtccttcgacggcgcgctcggcggcttcatcgagacgttcaacccgaacaacaacgctgccaacaagaccatcaacccttactggccgacgttcgactcgggcaagcagctcctcttcaacacgacgacgagggacaccctctctcccgccgacccgcgcatcgttgagacttcaagcttgaccgactttggcacgagccagaagaccaagtgcgacttctggcgtgggtcaatctcggtgaacgcgggtctc

SEQ ID NO: 2 - protein, 572 aa, Rhodosporidium toruloides
MLLNLFTLASLAATLQLAFASPTSLVRRTNPNEPPPVVDLGYARYQGYLNETAGLYWWRGIRYASAQRFQAPQTPATHKAVRNATEYGPICWPASEGTNTTKGLPPPSNSSSSAPQKQASEDCLFLNVVAPAGSCEGDNLPVLVYIHGGGYAFGDASTGSDFAAFTKHTGTKMVVVNLQYRLGSFGFLAGQAMKDYGVTNAGLLDQQFALQWVQQHVSKFGGNPDHVTIWGESAGAGSVMNQIIANGGNTVKALGLKKPLFHAAIGSSVFLPYQAKYNSPFAELLYSQLVSATNCTKAASSFACLEAVDAAALAAAGVKNSAAFPFGFWSYVPVVDGTFLTERASLLLAKGKKNLNGNLFTGINNLDEGFIFTDATIQNDTISDQSQRVSQFDRLLAGLFPYITSEERQAVAKQYPISDAPSKGNTFSRISAVIADSTFVCPTYWTAEAFGSSAHKGLFDYAPAHHATDNSYYIGSIWNGKKSVSSVQSFDGALGGFIETFNPNNNAANKTINPYWPTFDSGKQLLFNTTTRDTLSPADPRIVETSSLTDFGTSQKTKCDFWRGSISVNAGL

SEQ ID NO: 3 - DNA, 2220 bp, Rhodosporidium toruloides
ggatccacccgaactctgtcccgctttctggctttcttccttgctgtcgccccatcgcctttcccgactcgccgccatgctccttaacctcttcaccctcgcctccctcgctgcgacgctccagctcgcctttgcctctccgacctccctcgtccgccgcacgaacccaaacgagccccctcccgtcgtcgacctcggctacgcccgctaccaaggctacttgaacgagaccgccggactctactggtggcgcggaatccgctacgcctcggctcagcgcttccaggctcctcagacgcccgcgacgcacaaggccgtccgcaacgcgactgagtatggaccgatctgttggccggctagcgagggaaccaacacgaccaagggcttgccgccgcctagcaacagctcgagcagcgcgccgcagaaacaggcgtcggaggattgcctcttcctcaatgtcgttgcccccgccggctcgtgcgagggcgacaatcttcccgtcctcgtctacattcacggaggtggctacgccttcggcgatgcgagcaccggcagcgactttgccgccttcaccaagcacacgggaaccaagatggtcgttgtaaatctccagtaccgtctcggcagctttggtttcctcgctggccaagccatgaaggactacggtgtaacgaacgccggcttgcttgaccaggtgagtttcccgcatgatacccgcccacctttcgactcatgctgacgcctctcccgctcgcagcaattcgcccttcaatgggttcaacagcacgtctcgaagttcggcggcaaccccgatcacgttacgatttggggcgagtctgcaggcgcagggtccgttatgaaccagatcattgcgaacgtgagccacccgaaccgatctccagccgactttccccccccccccccccccgctgacctccctcgtcttgcagggcggcaacaccgtcaaggctctcggtctcaagaagcccctcttccacgctgccatcggctcctccgtcttcctcccctaccaagccaagtacaactcccccttcgccgagctgctctactcccaactcgtctcggcgacaaactgcaccaaagccgcctcgtccttcgcttgcctcgaagctgtcgacgctgcggcgctcgctgcggcgggcgtgaagaactcggcggcgttcccgttcgggttttggtcgtatgtcccggtcgtcgacgggaccttcttgactgagcgcgcgtcgctccttctcgccaagggcaagaagaacctcaatggcgtgcgtggcgagctttcgagtgcttcaggatctcgctgacactgtcgaccggctcgcagaacctcttcaccgggatcaacaacctcgacgaagatgagttcccgtcgacggctctgttcgcccagcgagactgacttgttcttttgcgaagattacgattcatattcactgacgccactattcagaacgacacgatcagcgaccagtcgcagcgcgtctcccagttcgaccgcctcctcgccggcctcttcccctacatcacctcggaggagcgccaggccgtcgcgaagcagtacccgatctccgacgcgccgtcaaagggcaacaccttctctcgcatctcggccgtcatcgcggactcgaccttcgtgtgcgttccccgtcgtcttctccgagtattccgctgacttcccgcttgcccgcagctgcccgacctactggaccgccgaggcgttcggctcgtccgcccacaagggcctcttcgactacgcgccggctcaccacgcgaccgacaactcgtactacatcggctccatctggaacggcaagaagtcggtctcgtccgtccagtccttcgacggcgcgctcggcggcttcatcgagacgttcaacccgaacaacaacgctgccaacaagaccatcaacccttactggccgacgttcgactcgggcaagcagctcctcttcaacacgacgacgagggacaccctctctcccgccgacccgcgcatcgttgagacttcaagcttgaccgactttggcacgagccagaagaccaagtgcgacttctggcgtgggtcaatctcggtgaacgcgggtctctaggcgtctttccttccgacttccttcgttctttcgttgtttattcttgcagttccgttgtatcggccattcgtgcgtgtagctcactcgagtatagacgttggcaagtgcgaaa

SEQ ID NO: 4 - protein, 544 aa, Rhodosporidium toruloides
TNPNEPPPVVDLGYARYQGYLNETAGLYWWRGIRYASAQRFQAPQTPATHKAVRNATEYGPICWPASEGTNTTKGLPPPSNSSSSAPQKQASEDCLFLNVVAPAGSCEGDNLPVLVYIHGGGYAFGDASTGSDFAAFTKHTGTKMVVVNLQYRLGSFGFLAGQAMKDYGVTNAGLLDQQFALQWVQQHVSKFGGNPDHVTIWGESAGAGSVMNQIIANGGNTVKALGLKKPLFHAAIGSSVFLPYQAKYNSPFAELLYSQLVSATNCTKAASSFACLEAVDAAALAAAGVKNSAAFPFGFWSYVPVVDGTFLTERASLLLAKGKKNLNGNLFTGINNLDEGFIFTDATIQNDTISDQSQRVSQFDRLLAGLFPYITSEERQAVAKQYPISDAPSKGNTFSRISAVIADSTFVCPTYWTAEAFGSSAHKGLFDYAPAHHATDNSYYIGSIWNGKKSVSSVQSFDGALGGFIETFNPNNNAANKTINPYWPTFDSGKQLLFNTTTRDTLSPADPRIVETSSLTDFGTSQKTKCDFWHGSISVNAGL

SEQ ID NO: 5 - protein, 15 aa, Rhodosporidium toruloides
TNPNEPPPVVDLGYA

SEQ ID NO: 6 - DNA, 24 bp, Other nucleic acid
gatcacccgggttgggcccactag

SEQ ID NO: 7 - DNA, 30 bp, Other nucleic acid
actcgccgccatggtccttaacctcttcac

SEQ ID NO: 8 - DNA, 30 bp, Other nucleic acid
gaaagacccctagagacccgcgttcaccga

SEQ ID NO: 9 - protein, 6 aa, Rhodosporidium toruloides
TNPNEP

SEQ ID NO: 10 - protein, 17 aa, Other nucleic acid
ACNAAYCCNAAYGARCC

SEQ ID NO: 11 - protein, 17 aa, Other nucleic acid
GGYTCRTTNGGRTTNGT

SEQ ID NO: 12 - protein, 17 aa, Other Nucleic Acid
GGYTCRTTGGGRTTNGT

SEQ ID NO: 13 - protein, 17 aa, Other nucleic acid
GGYTCRTTAGGRTTNGT

SEQ ID NO: 14 - protein, 17 aa, Other nucleic acid
GGYTCRTTTGGRTTNGT

SEQ ID NO: 15 - protein, 17 aa, Other nucleic acid
GGYTCRTTCGGRTTNGT

(19) United States
(12) Patent Application Publication
(10) Pub. No.: US 2002/0048781 A1
(43) Pub. Date: Apr. 25, 2002

(54) Direct production of desacetylcephalosporin C

(76) Inventors: Shu-Jen David Chiang, Manlius, NY (US); Jonathan David Basch, East Syracuse, NY (US)

Correspondence Address: MARLA J MATHIAS, BRISTOL-MYERS SQUIBB COMPANY, PATENT DEPARTMENT, P O BOX 4000, PRINCETON, NJ, 08543-4000, US

(21) Appl. No.: 09/801,852

(22) Filed: Mar. 8, 2001

Related U.S. Application Data
(60) Provisional application No. 60/188,033, filed on Mar. 9, 2000.

(51) Int. Cl. C12P 35/00

(52) U.S. Cl. 435/47

(57) ABSTRACT

The present invention concerns the direct production of desacetylcephalosporin C by culturing a strain of Acremonium chrysogenum containing recombinant nucleic acid encoding Rhodosporidium toruloides cephalosporin esterase.

This application is related to provisional U.S. patent application Ser. No. 60/188,033, filed Mar. 9, 2000, from which priority is claimed under 35 U.S.C. 119(e)(1), the disclosure of which is hereby incorporated by reference in its entirety.

### FIELD OF THE INVENTION

The present invention concerns the direct production of desacetylcephalosporin C by culturing a strain of Acremonium chrysogenum containing recombinant nucleic acid encoding Rhodosporidium toruloides cephalosporin esterase.

### BACKGROUND OF THE INVENTION

Cephalosporin C is a fermentation product of the fungal organism, Acremonium chrysogenum (formerly Cephalosporium acremonium). Cephalosporin C can be chemically converted to 7-aminocephalosporanic acid (7-ACA), the β-lactam nucleus used in the manufacture of semisynthetic cephalosporins. In fermentation broth, non-enzymatic breakdown of cephalosporin C to 2-(D-4-amino-4-carboxybutyl)-thiazole-4-carboxylic acid (compound X, FIG. 1) results in the loss of approximately 40% of the cephalosporin C produced (Usher et al., 1988, Biotechnol. Lett. 10, 543-548). Desacetylcephalosporin C, however, is far more resistant to this reaction. A cephalosporin esterase enzyme produced by Rhodosporidium toruloides can deacetylate cephalosporin C to form desacetylcephalosporin C.

U.S. Pat. No. 5,869,309 describes the cloning and sequencing of R. toruloides cephalosporin esterase genomic and cDNA genes. Heretofore, the expression of the cephalosporin esterase gene in A. chrysogenum in order to ferment desacetylcephalosporin C directly has been unknown.

### SUMMARY OF THE INVENTION

We have generated a recombinant fungal organism capable of fermenting desacetylcephalosporin C by transforming a cephalosporin esterase gene from Rhodosporidium toruloides into Acremonium chrysogenum (Cephalosporium acremonium). The cephalosporin esterase gene is expressed from a fungal promoter, preferably expressed from the promoter of the Aspergillus nidulans trpC gene under standard fermentation conditions for A. chrysogenum. The expression of an active cephalosporin esterase enzyme in A. chrysogenum results in the conversion of cephalosporin C to desacetylcephalosporin C in vivo, a novel fermentation process for the production of desacetylcephalosporin C. Thus, the present invention concerns a process for the direct production of desacetylcephalosporin C comprising culturing a strain of Acremonium chrysogenum containing nucleic acid encoding enzymes for cephalosporin C biosynthesis and recombinant nucleic acid encoding Rhodosporidium cephalosporin esterase under conditions suitable for the production of cephalosporin C and the expression of cephalosporin esterase such that the cephalosporin C so produced is converted to desacetylcephalosporin C.

### BRIEF DESCRIPTION OF THE DRAWINGS

FIG. 1. Conversion of cephalosporin C to desacetylcephalosporin C and chemical breakdown of cephalosporin C to 2-(D-4-amino-4-carboxybutyl)-thiazole-4-carboxylic acid (compound X).

FIG. 2. Preparation of plasmids pBMesterase1a and pBMesterase1b.

FIG. 3. Preparation of plasmid pBMesterase3.

FIG. 4. Preparation of plasmids pSJC62.3.

FIG. 5. Preparation of plasmid A.

FIG. 6. Preparation of plasmid pBMesterase11.

FIG. 7. The N-terminus of the protein (SEQ.I.D.NO.:9), the reverse translation sequence of the genomic N-terminus (SEQ.I.D.NO.:10), the inverse translation sequence that is complementary to the reverse translation sequence (SEQ.I.D.NO.:11), and the four oligonucleotide probes (Probes 1-4, SEQ.I.D.NOS.:12-15, respectively) used to identify the gene for the esterase.

FIGS. 8A and 8B. The cDNA sequence coding for an esterase useful in the present invention (SEQ. I.D. NO.:1) and the corresponding amino acid sequence of the esterase (SEQ. I.D. NO.:2).

FIGS. 9A and 9B. The genomic DNA sequence coding for an esterase useful in the invention (SEQ. I.D. NO.:3) and the corresponding amino acid sequence of the esterase (SEQ. I.D. NO.:2).

FIG. 10. The amino acid sequence of an esterase useful in the present invention containing 572 amino acids (SEQ. ID. NO.:2) showing the 544 amino acid sequence of the mature peptide (SEQ. ID. NO.:4) which typically has better enzymatic activity than the entire protein.

FIG. 11. Analysis of the amino acid composition of an intact esterase useful in the present invention.

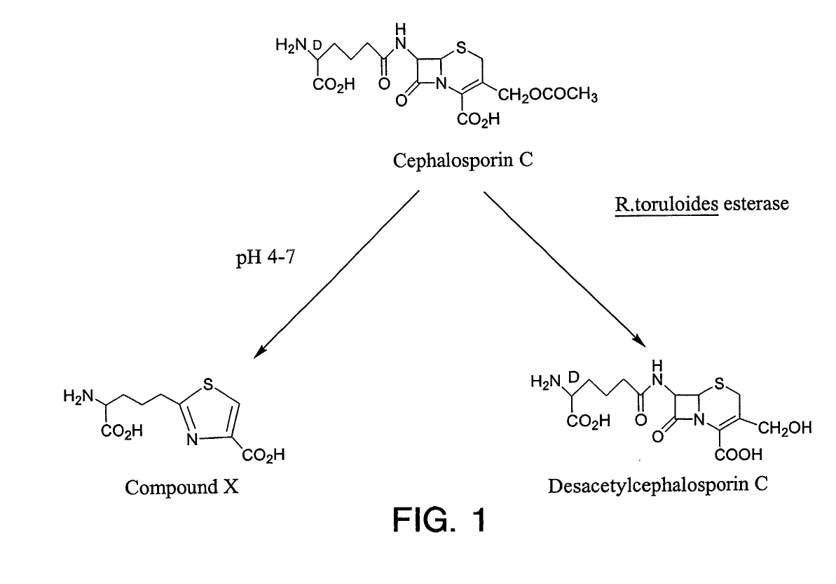

### DETAILED DESCRIPTION OF THE INVENTION

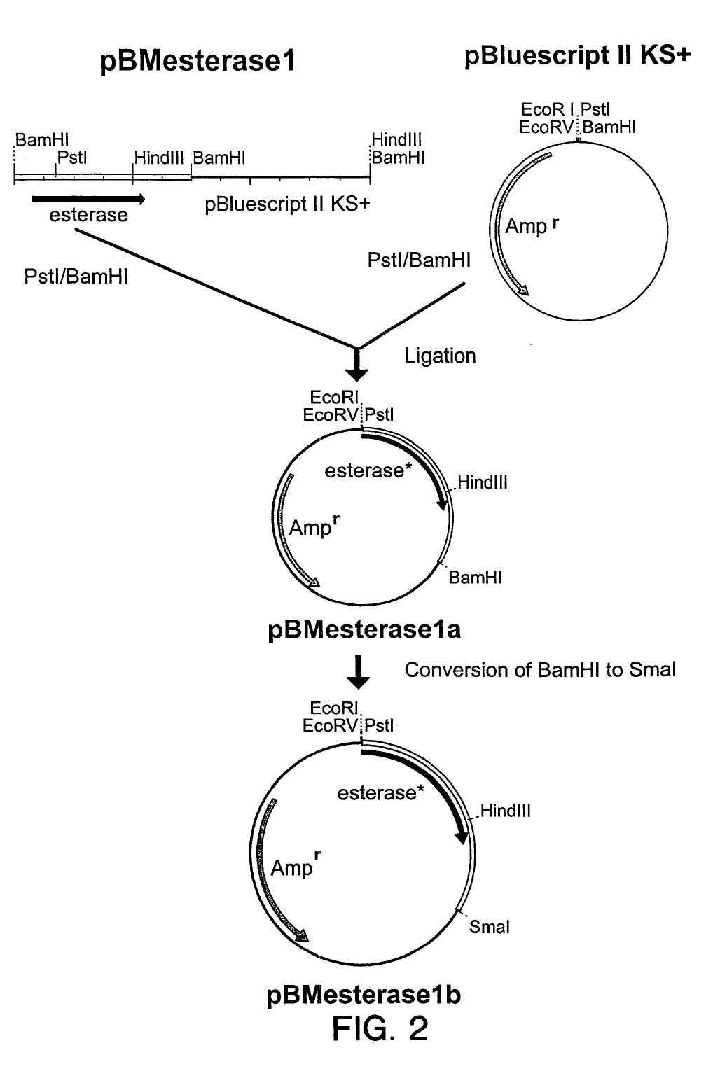

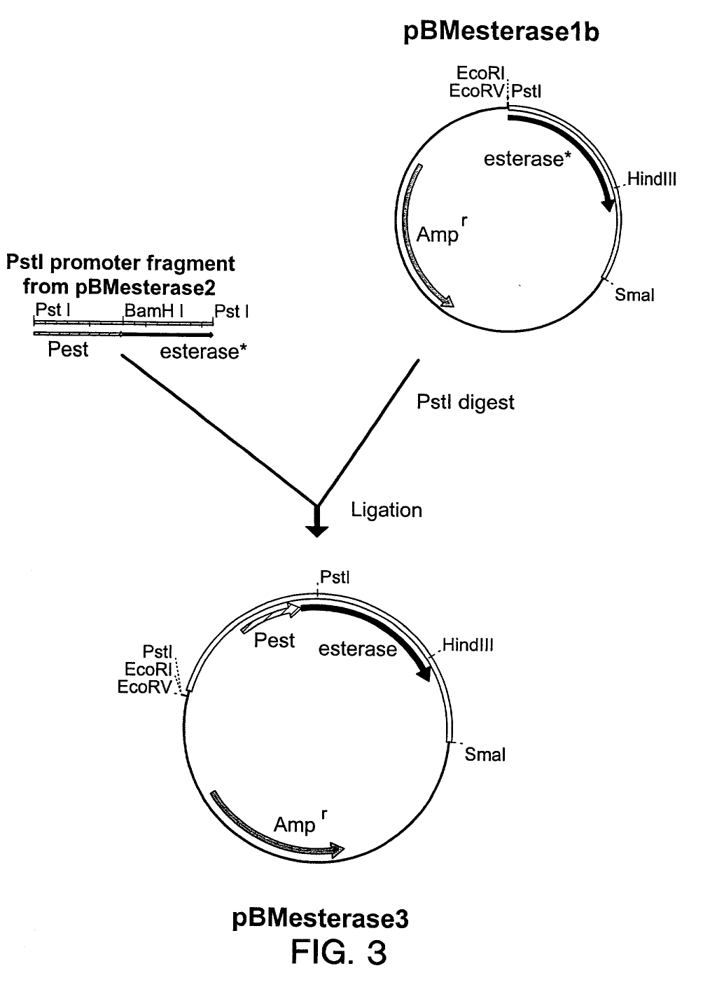

The present invention concerns a process for directly producing desacetylcephalosporin C using a host cell containing recombinant nucleic acid having a sequence coding for all or part of cephalosporin esterase from Rhodosporidium toruloides. A preferred source of the esterase nucleic acid is Rhodosporidium toruloides ATCC 10657 which is well known in the art and is deposited with and available from the American Type Culture Collection, Rockville, Md. and is described in U.S. Pat. No. 4,533,632. Preferably, the recombinant nucleic acid molecule is a DNA molecule and the nucleic acid sequence is a DNA sequence. All DNA sequences are represented herein by formulas whose left to right orientation is in the conventional direction of 5′ to 3′. Nucleotide base abbreviations used herein are conventional in the art, i.e., T is thymine, A is adenine, C is cytosine, and G is guanine; also, X is A, T, C, or G, Pu is purine (i.e., G or A), and Py is pyrimidine (i.e., T or G). Further preferred as the DNA for the recombinant esterase is a DNA sequence having all or part of the nucleotide sequence substantially as shown in FIGS. 2 and 3; or a DNA sequence complementary to one of these DNA sequences; or a DNA sequence which hybridizes to a DNA sequence complementary to one of these DNA sequences. Preferably, the DNA sequence hybridizes under stringent conditions. Stringent hybridization conditions select for DNA sequences of greater than 80% identity, preferably greater than 85% or, more preferably, greater than 90% identity. Screening DNA under stringent conditions may be carried out according to the method described in Nature 313, 402-404 (1985). The DNA sequences capable of hybridizing under stringent conditions with the DNA disclosed in the present application may be, for example, allelic variants of the disclosed DNA sequences, may be naturally present in Rhodosporidium toruloides but related to the disclosed DNA sequences, or may be derived from other bacterial, fungal or yeast sources. General techniques of nucleic acid hybridization are disclosed by Sambrook et al., In: Molecular Cloning, A Laboratory Manual, 2nd edition, Cold Spring Harbor Laboratory, Cold Spring Harbor, N.Y. (1989), and by Haymes et al., In: Nucleic Acid Hybridization, A Practical Approach, IRL Press, Washington, D.C. (1985), which references are incorporated herein by reference. In the case of a nucleotide sequence (e.g., a DNA sequence) coding for part of cephalosporin esterase, it is required that the nucleotide sequence code for a fragment that is or can be processed to be catalytically active, i.e., has esterase activity.

It is also contemplated that the recombinant DNA useful in the present invention encompasses modified sequences. As used in the present application, the term “modified”, when referring to a nucleotide or polypeptide sequence, means a nucleotide or polypeptide sequence which differs from the wild-type sequence found in nature.

The recombinant DNA sequences useful in the present invention can be obtained using various methods well-known to those of ordinary skill in the art. At least three alternative principal methods may be employed:

(1) the isolation of a double-stranded DNA sequence from genomic DNA or complementary DNA (cDNA) which contains the sequence;

(2) the chemical synthesis of the DNA sequence; and

(3) the synthesis of the DNA sequence by polymerase chain reaction (PCR).

In the first approach, a genomic or cDNA library can be screened in order to identify a DNA sequence coding for all or part of cephalosporin esterase. For example, a R. toruloides genomic DNA library can be screened in order to identify the DNA sequence coding for all or part of cephalosporin esterase. Various techniques can be used to screen the genomic DNA or cDNA libraries.

For example, labeled single stranded DNA probe sequences duplicating a sequence present in the target genomic DNA or cDNA coding for all or part of cephalosporin esterase can be employed in DNA/DNA hybridization procedures carried out on cloned copies of the genomic DNA or cDNA which have been denatured to single stranded form.

A genomic DNA or cDNA library can also be screened for a genomic DNA or cDNA coding for all or part of cephalosporin esterase using immunoblotting techniques.

In one typical screening method suitable for either immunoblotting or hybridization techniques, the genomic DNA library, which is usually contained in a vector, or cDNA library is first spread out on agar plates, and then the clones are transferred to filter membranes, for example, nitrocellulose membranes. A DNA probe can then be hybridized or an antibody can then be bound to the clones to identify those clones containing the genomic DNA or cDNA coding for all or part of cephalosporin esterase.

In the second approach, the DNA sequences of the present invention coding for all or part of cephalosporin esterase can be chemically synthesized. For example, the DNA sequence coding for cephalosporin esterase can be synthesized as a series of 100 base oligonucleotides that can be sequentially ligated (via appropriate terminal restriction sites or complementary terminal sequences) so as to form the correct linear sequence of nucleotides.

In the third approach, the DNA sequences of the present invention coding for all or part of cephalosporin esterase can be synthesized using PCR. Briefly, pairs of synthetic DNA oligonucleotides at least 15 bases in length (PCR primers) that hybridize to opposite strands of the target DNA sequence are used to enzymatically amplify the intervening region of DNA on the target sequence. Repeated cycles of heat denaturation of the template, annealing of the primers and extension of the 3′-termini of the annealed primers with a DNA polymerase results in amplification of the segment defined by the 5′ ends of the PCR primers. See, White et al., Trends Genet. 5, 185-189 (1989).

The recombinant DNA sequences useful in the present invention coding for all or part of cephalosporin esterase can also be modified (i.e., mutated) to prepare various mutations. Such mutations may be either degenerate, i.e., the mutation changes the amino acid sequence encoded by the mutated codon, or non-degenerate, i.e., the mutation does not change the amino acid sequence encoded by the mutated codon. These modified DNA sequences may be prepared, for example, by mutating the cephalosporin esterase DNA sequence so that the mutation results in the deletion, substitution, insertion, inversion or addition of one or more amino acids in the encoded polypeptide using various methods known in the art. For example, the methods of site-directed mutagenesis described in Morinaga et al., Bio/Technol. 2, 636-639 (1984), Taylor et al., Nucl. Acids Res. 13, 8749-8764 (1985) and Kunkel, Proc. Natl. Acad. Sci. USA 82, 482-492 (1985) may be employed. In addition, kits for site-directed mutagenesis may be purchased from commercial vendors. For example, a kit for performing site-directed mutagenesis may be purchased from Amersham Corp. (Arlington Heights, Ill.). In addition, disruption, deletion and truncation methods as described in Sayers et al., Nucl. Acids Res. 16, 791-802 (1988) may also be employed. Both degenerate and non-degenerate mutations may be advantageous in producing or using the polypeptides of the present invention. For example, these mutations may permit higher levels of production, easier purification, or provide additional restriction endonuclease recognition sites. All such modified DNA and polypeptide molecules are contemplated to be useful in the present invention.

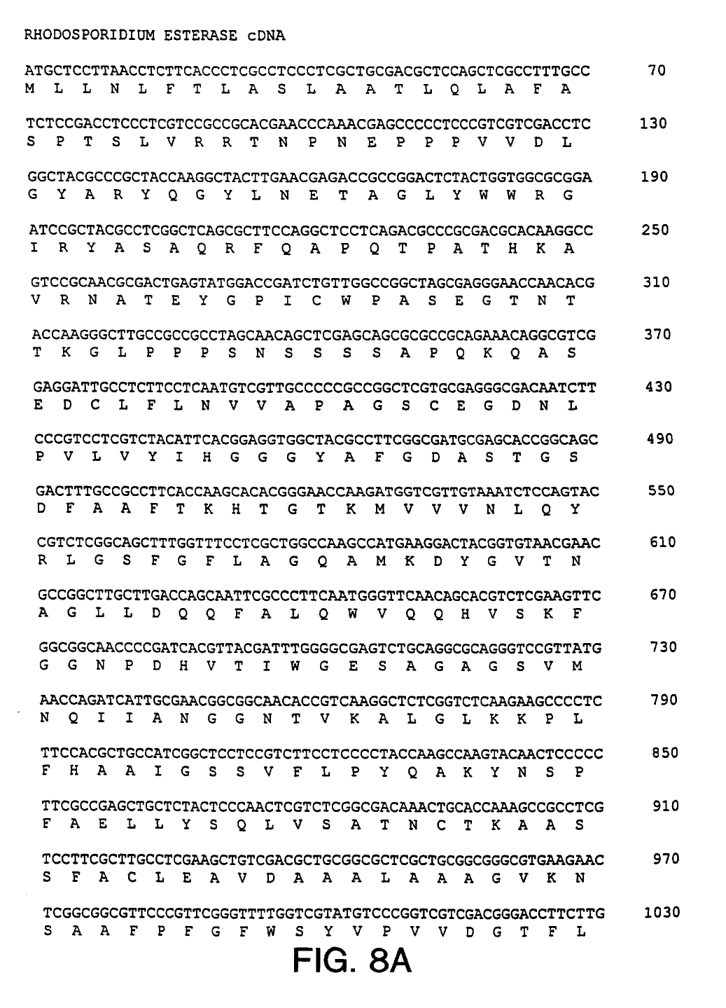

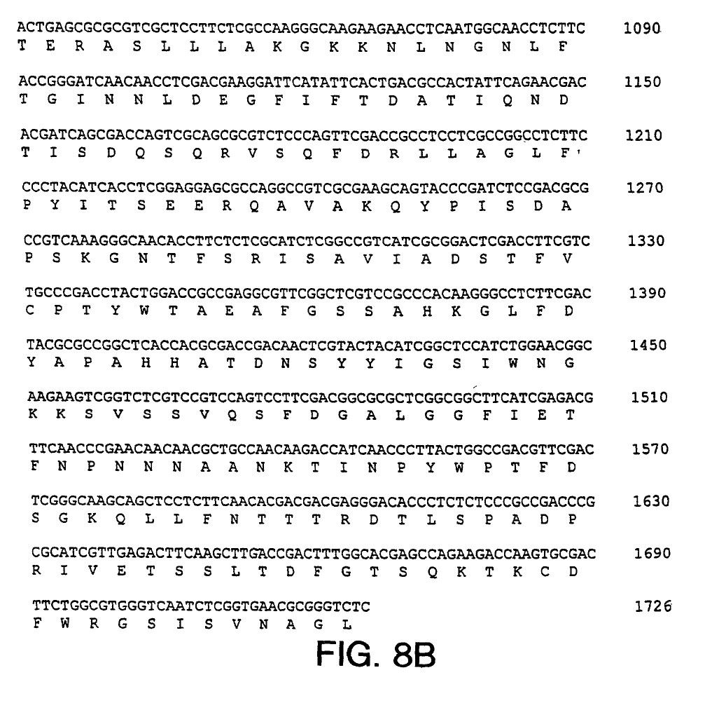

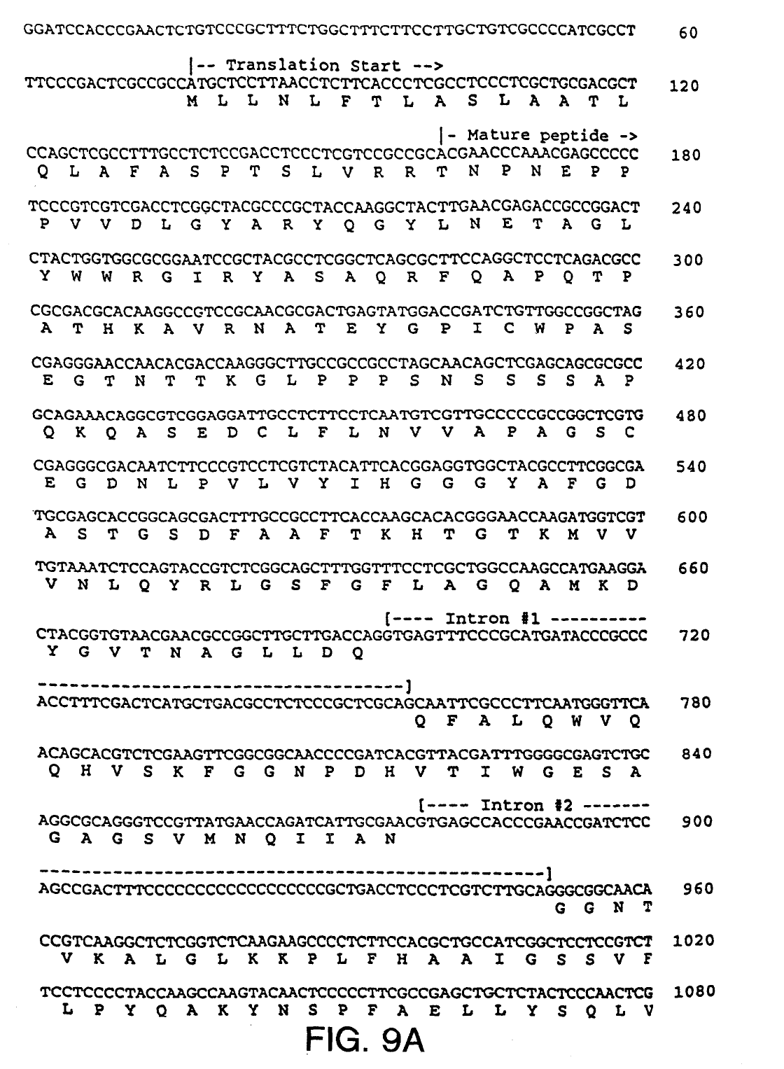

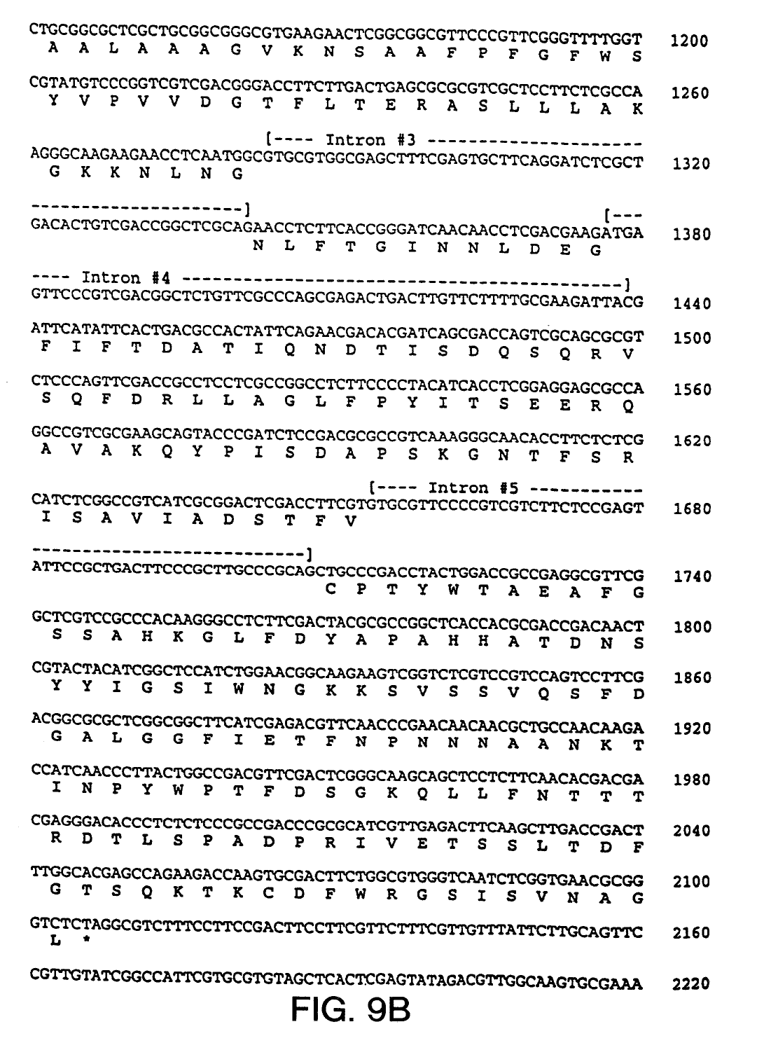

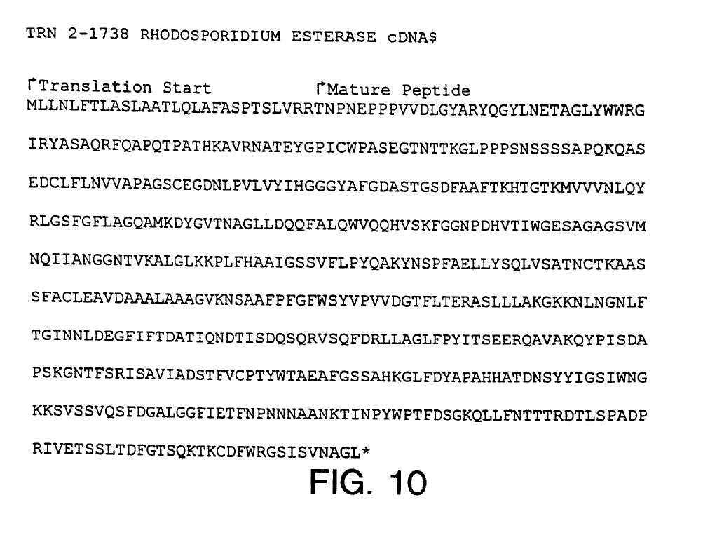

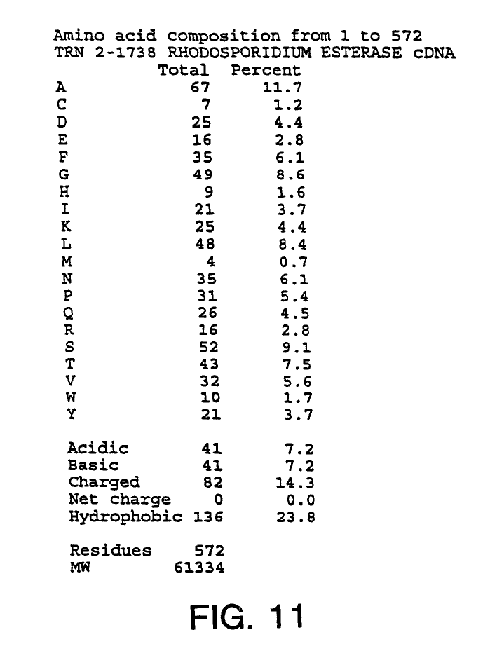

The A. chrysogenum host cells useful in the process of the invention contain expression vectors comprising a DNA sequence coding for all or part of cephalosporin esterase. The expression vectors preferably contain all or part of one of the DNA sequences having the nucleotide sequences substantially as shown in FIGS. 8 or 9. Further preferred are expression vectors comprising one or more regulatory DNA sequences operatively linked to the DNA sequence coding for all or part of cephalosporin esterase. As used in this context, the term “operatively linked” means that the regulatory DNA sequences are capable of directing the replication and/or the expression of the DNA sequence coding for all or part of cephalosporin esterase.

Expression vectors of utility in the present invention are often in the form of “plasmids”, which refer to circular double stranded DNA loops which, in their vector form, are not bound to the chromosome. However, the invention is intended to include such other forms of expression vectors which serve equivalent functions and which become known in the art subsequently hereto.

Expression vectors useful in the present invention typically contain an origin of replication, a promoter located in front (i.e., upstream of) the DNA sequence and followed by the DNA sequence coding for all or part of cephalosporin esterase. The DNA sequence coding for all or part of the structural protein is followed by transcription termination sequences and the remaining vector. The expression vectors may also include other DNA sequences known in the art, for example, stability leader sequences which provide for stability of the expression product, secretory leader sequences which provide for secretion of the expression product, sequences which allow expression of the structural gene to modulated (e.g., by the presence or absence of nutrients or other inducers in the growth medium), marking sequences which are capable of providing phenotypic selection in transformed host cells, stability elements such as centromeres which provide mitotic stability to the plasmid, and sequences which provide sites for cleavage by restriction endonucleases. The characteristics of the actual expression vector used must be compatible with the host cell which is to be employed. For example, when cloning in a fungal cell system, the expression vector should contain promoters isolated from the genome of fungal cells (e.g., the cephalosporin esterase promoter from R. toruloides or the trpC promoter from Aspergillus nidulans). Certain expression vectors may contain a fungal autonomously replicating sequence (ARS; e.g., ARS from Fusarium oxysporum and Saccharomyces cerevisiae) which promotes in vivo production of self-replicating plasmids in fungal hosts. It is preferred that the fungal expression vectors of the invention do not have a fungal ARS sequence and thus will integrate into host chromosomes upon plasmid entry of host cells. Such integration is preferred because of enhanced genetic stability. An expression vector as contemplated for use in the present invention is at least capable of directing the replication in Escherichia coli and integration in fungal cells, and preferably the expression, of the cephalosporin esterase DNA sequences of the present invention. Suitable origins of replication in various E. coli hosts include, for example, a ColEl plasmid replication origin. Suitable promoters include, for example, the trpC promoter from A. nidulans and the neo-r gene promoter from E. coli. Suitable termination sequences include, for example, the trpC terminator from A. nidulans, and the neo-r gene terminator from E. coli. It is also preferred that the expression vectors include a sequence coding for a selectable marker. The selectable marker is preferably antibiotic resistance. As selectable markers, phleomycin resistance (for fungal cells), ampicillin resistance, and neomycin resistance (for bacterial cells) can be conveniently employed. All of these materials are known in the art and are commercially available.

Suitable expression vectors containing the desired coding and control sequences may be constructed using standard recombinant DNA techniques known in the art, many of which are described in Sambrook et al. Molecular Cloning: A Laboratory Manual, 2nd edition, Cold Spring Harbor Laboratory, Cold Spring Harbor, N.Y. (1989). Preferred plasmids are pSJC62.3 and pBMesterase11 described herein.

The A. chrysogenum host cells containing an expression vector which comprises a DNA sequence coding for all or part of cephalosporin esterase also contain nucleic acid (preferably DNA) encoding enzymes for cephalosporin C biosynthesis. The DNA encoding the enzymes for cephalosporin C biosynthesis is typically endogenous in A. chrysogenum strains; however, host cells engineered to contain nucleic acid encoding enzymes for cephalosporin C biosynthesis are also contemplated to be within the scope of the present invention. Examples of host cells which can be transformed according to the present invention include A. chrysogenum strains ATCC 11550, ATCC 36225, ATCC 48272 and their derivatives developed by various industrial strain improvement programs.

The host cells preferably contain an expression vector which comprises all or part of one of the DNA sequence having the nucleotide sequences substantially as shown in FIGS. 8 or 9. Further preferred are host cells containing an expression vector comprising one or more regulatory DNA sequences capable of directing the replication and/or the expression of and operatively linked to a DNA sequence coding for all or part of cephalosporin esterase.

Expression vectors may be introduced into host cells by various methods known in the art. For example, transformation of host cells with expression vectors can be carried out by the polyethylene glycol mediated protoplast transformation method. However, other methods for introducing expression vectors into host cells, for example, electroporation, biolistic injection, or protoplast fusion, can also be employed.

Once an expression vector has been introduced into an appropriate host cell, the host cell may be cultured under conditions permitting production of cephalosporin C and expression of cephalosporin esterase, which result in the conversion of cephalosporin C to desacetylcephalosporin C in vivo.

A novel transformant of the type described above, comprising an A. chrysogenum host cell transformed with the recombinant DNA expression vector plasmid pBMesterase11 integrated into the chromosomal DNA of said host cell, and identified as DC11, has been deposited with the American Type Culture Collection, Rockville, Md., on Jan. 27, 1999, under the Budapest Treaty and assigned ATCC accession no. 74482.

Host cells containing an expression vector which contains a DNA sequence coding for all or part of cephalosporin esterase may be identified by one or more of the following six general approaches: (a) DNA-DNA hybridization; (b) the presence or absence of marker gene functions; (c) assessing the level of gene expression as measured by the production of cephalosporin esterase mRNA transcripts in the host cell; (d) enzyme assay; (e) colorimetric detection; and (f) detection of the end product of the expressed cephalosporin esterase in fermentation, e.g., desacetylcephalosporin C, detection of the end product being the preferred method of identification.

In the first approach, the presence of a DNA sequence coding for all or part of cephalosporin esterase can be detected by DNA-DNA or RNA-DNA hybridization using probes complementary to the DNA sequence.

In the second approach, the recombinant expression vector host system can be identified and selected based upon the presence or absence of certain marker gene functions (e.g., acetamide utilization, resistance to antibiotics, resistance to fungicide, uracil prototrophy, etc.). A marker gene can be placed in the same plasmid as the DNA sequence coding for all or part of cephalosporin esterase under the regulation of the same or a different promoter used to regulate the cephalosporin esterase coding sequence. Expression of the marker gene in response to induction or selection indicates the presence of the entire recombinant expression vector which carries the DNA sequence coding for all or part of cephalosporin esterase. Alternatively, a marker gene can be placed in a different plasmid as the cephalosporin esterase gene and both plasmids cotransformed into A. chrysogenum (Menne et al.,1994, Appl. Microbiol. Biotechnol. 42, 27-35).

In the third approach, the production of cephalosporin esterase mRNA transcripts can be assessed by hybridization assays. For example, polyadenylated RNA can be isolated and analyzed by Northern blotting or reverse transcription PCR (RT-PCR) assay using a probe complementary to the RNA sequence. Alternatively, the total RNA of the host cell may be extracted and assayed for hybridization to such probes.

In the fourth approach, expression of cephalosporin esterase can be measured by assaying for cephalosporin esterase enzyme activity using known methods. For example, the assay described in the Examples section hereof may be employed.

In the fifth approach, the expression of cephalosporin esterase protein can also be assessed by colorimetric detection. For example, in cells known to be deficient in this enzyme, expression of cephalosporin esterase activity can be detected on the enzymatic hydrolysis of a colorless substrate, p-nitrophenyl acetate, to a yellow colored p-nitrophenylate on the media plate.

In the sixth approach, the expression of cephalosporin esterase can be further assessed by the conversion of cephalosporin C to desacetylcephalosporin C in fermentation broth. For example, the concentration of cephalosporin C and desacetylcephalosporin C in the fermentation broth can be determined by high performance liquid chromatography (HPLC) on a reverse-phase column (Usher et al, 1985, Anal. Biochem. 149,105-110).

The DNA sequence of expression vectors, plasmids or DNA molecules useful in the present invention may be determined by various methods known in the art. For example, the dideoxy chain termination method as described in Sanger et al., Proc. Natl. Acad. Sci. USA 74, 5463-5467 (1977), or the Maxam-Gilbert method as described in Proc. Natl. Acad. Sci. USA 74, 560-564 (1977) may be employed.

It should, of course, be understood that not all expression vectors and DNA regulatory sequences will function equally well to express the DNA sequences useful in the present invention. Neither will all host cells function equally well with the same expression system. However, one of ordinary skill in the art may make a selection among expression vectors, DNA regulatory sequences, and host cells using the guidance provided herein without undue experimentation and without departing from the scope of the present invention.

All amino acid residues identified herein are in the natural L-configuration. In keeping with standard polypeptide nomenclature, J. Biol. Chem. 243, 3557-3559 (1969), abbreviations for amino acid residues are as shown in the following Table of Correspondence:

TABLE OF CORRESPONDENCE

SYMBOL

| 1-Letter | 3-Letter | AMINO ACID |
|---|---|---|
| Y | Tyr | L-tyrosine |
| G | Gly | L-glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

All amino acid sequences are represented herein by formulas whose left to right orientation is in the conventional direction of amino-terminus to carboxyl-terminus.

The desacetylcephalosporin C produced by the process of the invention may be isolated and purified to some degree using various protein purification techniques. For example, chromatographic procedures such as ion exchange chromatography, gel filtration chromatography and immunoaffinity chromatography may be employed.

The polypeptides described herein have been defined by means of determined DNA and deduced amino acid sequencing. Due to the degenerate nature of the genetic code, which results from there being more than one codon for most of the amino acid residues and stop signals, other DNA sequences which encode the same amino acid sequence may be used for the production of the polypeptide of the present invention. In addition, it will be understood that allelic variations of these DNA and amino acid sequences naturally exist, or may be intentionally introduced using methods known in the art. These variations may be demonstrated by one or more amino acid differences in the overall sequence, or by deletions, substitutions, insertions, inversions or additions of one or more amino acids in said sequence. Such amino acid substitutions may be made, for example, on the basis of similarity in polarity, charge, solubility, hydrophobicity, hydrophilicity and/or the amphiphatic nature of the residues involved. For example, negatively charged amino acids include aspartic acid and glutamic acid; positively charged amino acids include lysine and arginine; amino acids with uncharged polar head groups or nonpolar head groups having similar hydrophilicity values include the following: leucine, isoleucine, valine, glycine, alanine, asparagine, glutamine, serine, threonine, phenylalanine, tyrosine. Other contemplated variations include salts and esters of the aforementioned polypeptides, as well as precursors of the aforementioned polypeptides, for example, precursors having N-terminal substituents such as methionine, N-formyl-methionine used and leader sequences. All such variations are included within the scope of the present invention.

As used herein the term “culturing” means incubating the organisms in a medium such that the desired polypeptides are produced, e.g., actively growing the cells in a growth medium. The process of the invention is in situ fermentation and conversion (single-stage fermentation and conversion). The process of the present invention is performed under conditions suitable for production of the desired desacetylcephalosporin C. It is preferred to employ an aqueous liquid as the reaction (culture) medium, although an organic liquid, or a miscible or immiscible (biphasic) organic/aqueous liquid mixture may also be employed.

Culturing the A. chrysogenum host cells may be achieved by one of ordinary skill in the art by the use of an appropriate medium. Appropriate media for growing host cells include those which provide nutrients necessary for the growth of the cells. A typical medium for growth includes necessary carbon sources, nitrogen sources, and trace elements. Inducers may also be added. The term “inducer”, as used herein, includes any compound enhancing formation of the desired protein, peptide or antibiotic.

Carbon sources may include sugars such as maltose, lactose, glucose, fructose, glycerol, cerelose, sorbitol, sucrose, starch, mannitol, galactose, raffinose, and the like; organic acids such as sodium acetate, sodium citrate, and the like; amino acids such as lysine, sodium glutamate, and the like.

Nitrogen sources may include N-Z amine A, corn steep liquor, soy bean meal, beef extracts, yeast extracts, malt extracts, casamino acids, yeastamin, molasses, baker's yeast, tryptone, soyflour, peptone, Pharmamedia, sodium nitrate, ammonium sulfate, and the like.

Trace elements may include phosphates, magnesium, zinc, copper manganese, calcium, cobalt, nickel, iron, sodium, potassium salts, and the like.

The medium employed may include more than one carbon or nitrogen source or other nutrient.

A preferred fermentation medium comprises 5-15% corn steep liquor, 1-6% soyflour, 1-6% Pharmamedia, 1-6% glucose, 0.1-1.0% CaSO₄, 0.1-1.0% KH₂PO₄, 0.1-1.0% MgSO₄, 7H₂O,1 0.1-1.0% (NH₄)₂SO₄, 0.5-2.0% methionine, and 1-5% lard oil. The pH of the fermentation medium is preferably adjusted to about 5.5 to 7.5, more preferably about 6.2 to 7.0.

Sometimes it may be desirable to use a seed medium. A “seed medium” differs from a normal fermentation medium in that readily available carbon and nitrogen sources are used to promote a fast increase of total cell mass. Usually, some of the inducers are not included in the seed medium. A preferred seed medium comprises 1-10% corn steep liquor, 2-10% glucose, 2-10% Pharmamedia, 0.1-1.0%(NH₄)₂SO₄, 0.5-2.0% CaCO₃, and 0.001-0.01% ZnSO₄, 7H₂O. The pH of the seed medium is preferably adjusted to about 6.0 to 7.5, more preferably about 6.5 to 7.2.

The pH of the fermentation medium is preferably adjusted to about 5.5 to 7.5, depending upon the particular medium, sterilized, e.g., at a temperature of 121° C. for 30 minutes, and then adjusted to a desirable pH, after sterilization. The pH of the medium during growth of the host cells is most preferably maintained between about 6.2 and 7.0, during the vegetative cell growth phase, and most preferably between about 5.7 and 6.5, during the desacetylcephalosporin C production phase. A suitable temperature range for the process of the invention is from about 22° C. to about 29° C., most preferably about 25° C. to about 29° C. during the vegetative cell growth phase, and most preferably about 22° C. to about 26° C. during the desacetylcephalosporin C production phase.

Pressure is not known to be critical to practice of the invention and for convenience about atmospheric pressure is typically employed.

When growing host cells, the process of the invention is preferably carried out under aerobic conditions. The agitation and aeration of the reaction mixture affects the amount of oxygen available during the stereoselective reduction process which may be conducted, for example, in shake-flask cultures or fermentors during growth of microorganisms in a single-stage or two-stage process. The agitation range from 200 to 1,000 RPM is preferable, with 400 to 800 RPM being most preferred. Aeration of about 0.1 to 10 volumes of air per volume of media per minute (i.e., 0.1 to 10 vvm) is preferred, with aeration of about 5 volumes of air per volume of media per minute (i.e., 5 vvm) being most preferred.

After the initial 24-48 hours of cell growth phase, it is preferred to feed the fermentors with various amounts of (NH₄)₂SO₄, glucose and lard oil to allow the optimal condition for desacetylcephalosporin C production during the remaining fermentation period. Satisfactory production of desacetylcephalosporin C may take, for example, from about 72 to 240 hours, preferably 144 to 192 hours.

In the process of the present invention it is preferred that chemical breakdown of expressed cephalosporin C to 2-(D-4-amino-4-carboxybutyl)-thiazole-4-carboxylic acid is less than 40%, more preferably less than 30%, even more preferably less than 20%, even more preferably less than 10%, and most preferably less than 5%.

The product of the process of the present invention, i.e., desacetylcephalosporin C, may be isolated and purified by known methodologies such as by extraction distillation, crystallization, column chromatography, and the like.

A preferred method for separating the desired compound of desacetylcephalosporin C from the remaining compounds of the fermentation medium is concentration by removal of water, then extraction by absorption chromatography.

The following examples are further illustrative of the present invention. These examples are not intended to limit the scope of the present invention, and provide further understanding of the invention.

In the following examples, some reagents, plasmids, restriction enzymes and other materials were obtained from commercial sources and used according to the indication by suppliers. Operations employed for the purification and characterization and the cloning of DNA and the like are well known in the art or can be adapted from the literature.

### EXAMPLE 1

### Purification of Cephalosporin Esterase

1.1 Culture of Microorganism

Rhodosporidium toruloides (ATCC 10657) seed culture was initiated from the inoculation of frozen preservation cultures of 2% into 500 ml Erlenmeyer flasks containing 100 ml of the following medium: 2% glucose, 1% yeast extract, 1% Bacto-peptone, 0.5% KH₂PO₄, pH 6.0. Seed flasks were cultured for 24 hours at 28° C. and 250 rpm; 2% inoculum volume was used to start production stage fermentation. Production stage medium was composed of: 8% corn steep liquor, 1% KH₂PO₄, 3% glucose, pH 6.2. The media was autoclaved for two hours. This led to increased titers when compared to the normal autoclave time of 30 minutes. Fermentor broth was cultured for 3 or 4 days to 16-21° C. with high aeration. Specific activities of whole broth were typically in the range of 20-37 IU/ml.

1.2 Purification of the Enzyme From Rhodosporidium toruloides

The esterase was released from Rhodosporidium toruloides cells by treatment of the fermentation broth with 100 mM EDTA at pH 4.0 for 8 hours. Approximately 50% of the enzymatic activity could be released from the cells in this manner. The broth was centrifuged at 5,000×g to remove the cells and the corn steep solids. The supernatant was ultrafiltered through an Amicon hollow fiber cartridge with a molecular weight cut-off of 30,000 to 10% of the original volume. The enzyme was brought up to the original volume by addition of deionized water. The pH was brought up to 7.0 by addition of 2 M ammonium hydroxide and the enzyme solution added to DEAE Trisacryl (100 g resin/50 ml enzyme solution) which had been washed with 50 mM potassium phosphate buffer 7.0. The enzyme does not bind to DEAE and was obtained in the filtrate which was then brought to pH 4.5 with 1.0 M acetic acid. This solution was then loaded onto a carboxymethyl Sepharose column (18×3 cm) and washed with 50 mM ammonium acetate pH 4.5 until the absorbance at 280 nm was less than 0.1 (approximately 4 column volumes). The esterase was eluted with a linear gradient of 50 to 500 mM ammonium acetate pH 6.5 (flow rate 1.0 ml/min). Fractions of 7.0 ml were collected and the fractions containing esterase were pooled and concentrated on a 50,000 molecular weight cut off Centricon.

### EXAMPLE 2

### Characterization of Cephalosporin Esterase

2.1 Specific Activity of Enzyme

Enzyme was added to the reaction mixture containing the potassium salt of the cephalosporin (25-400 mM), 100 mM potassium phosphate, pH 6.5 in a final volume of 0.5 ml. The mixture was incubated at 30° C. (unless described otherwise) and stopped by addition of 2.0 ml 50% acetonitrile. The reaction was monitored at 254 nm by HPLC on a 5 micron C18 column (50×4 mm) with the mobile phase consisting of 25 mM octane sulfonic acid, 0.1% phosphoric acid, 12% methanol, pH 2.5. Protein was assayed using the Bio-Rad protein assay kit (Bio-Rad Co., USA) using bovine serum albumin as the standard. The enzyme exhibited Michaelis-Menton kinetics with cephalosporin C. From double reciprocal plots, the Km for hydrolysis of cephalosporin C was found to be 51.8 mM with a corresponding Vₘₐₓ of 77.0 μmole/min/mg. The reaction products, desacetylcephalosporin C and acetate did not inhibit the reaction to any appreciable extent. A 1.0% solution of cephalosporin C was completely hydrolyzed within 30 minutes at 30° C. with no side products observed by HPLC.

2.2 Substrate File

Esterase activity was measured using p-nitrophenyl ester substrates as well as cephalosporin derivatives. The enzyme was incubated at 30° C. (unless described otherwise) with p-nitrophenyl acetate, 10.0 mM in 100 mM potassium phosphate buffer pH 6.5 or 10.0 mM p-nitrophenyl esters ranging in carbon chain length from C:2 to C:18 in 100 mM potassium phosphate pH 6.5 and 2% acetonitrile. Enzyme activity was monitored spectrophotometrically by measuring the increase in absorbance at 405 nm due to the formation of the p-nitrophenylate ion. The assay for cephalosporin derivatives was as described in Example 2.1. The results are described in Table 1 for p-nitrophenyl ester substrates and Table 2 for cephalosporin derivatives.

TABLE 1
Effect of Increasing Ester Chain
Length on Esterase Activity.

| Length of Ester | Relative Activity (%) |
|---|---|
| Acetate C:2 | 100 |
| Propionate C:3 | 34 |
| Butyrate C:4 | 5 |
| Caproate C:6 | 0 |
| Caprylate C:8 | 0 |
| Caprate C:10 | 0 |
| Laurate C:12 | 0 |
| Myristate C:14 | 0 |
| Palmitate C:16 | 0 |
| Stearate C:18 | 0 |

TABLE 2
Relative rates of esterase activity against cephem substrates

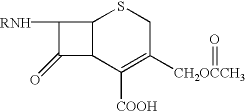

| Substrate | Relative Rate |
|---|---|
| R = —H | 100 |
| 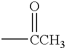 | 51 |
| 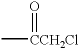 | 105 |
| 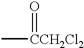 | 108 |
| 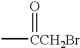 | 114 |
| 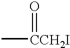 | 103 |
| 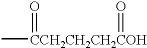 | 105 |
| 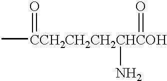 | 68 |
| 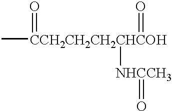 | 42 |
| 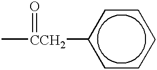 | 17 |
| 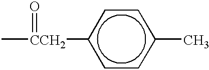 | 68 |
| 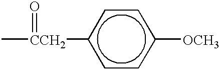 | 41 |
| 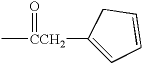 | 34 |

2.3 Effect of Temperature

A. Optimum Temperature

Enzyme was incubated with 10.0 mM p-nitrophenyl acetate in 100 mM potassium phosphate buffer pH 6.5. The reaction mixtures were incubated for 10 minutes in a shaking water bath at 300 rpm and at temperatures from 10 to 65° C. The optimal temperature for the reaction was 25° C.

B. Thermal Stability

Enzyme was incubated with p-nitrophenyl acetate as described in Example 2.3A. Enzyme was incubated at various temperatures for 15 minutes then immediately placed on ice. The enzyme was unstable when incubated at temperatures about 25° C. with rapid inactivation between 30 and 45° C.

2.4 Effect of pH

Enzyme was incubated with p-nitrophenyl acetate as described in Example 2.3A. A 100 mM Tris-maleate universal buffer with a pH range of 4 to 8 was used. The esterase was found to be active in a pH range of 4.5 to 7 with optimal activity at a pH of 6.0 with both p-nitrophenyl acetate and cephalosporin C.

2.5 Effect of Various Enzyme Modulators

Enzyme was incubated in the presence of 10 mM reagent for 15 minutes at 25° C. The reaction mixture was then diluted 100 fold into assay mix and assayed with p-nitrophenyl acetate. The results strongly suggest the presence of an active-site serine for the Rhodosporidium enzyme. Phenylmethylsulfonyl fluoride (PMSF), 3,4-dichloroisocoumarin (DCI), and dimethyl phosphite all inhibited the enzyme. The histidine-modifying reagent diethylpyrocarbonate essentially inactivated the enzyme. Sulfhydryl-modifying agents iodoacetamide and N-ethylmaleimide had little or no effect on the activity of the enzyme although slight activation was observed with β-mercaptoethanol and dithiothreitol. The presence or absence of metal ions also had little or no effect on the enzyme although slight inhibition was observed with EDTA.

2.6 Determination of Isoelectric Point (pl)

Isoelectric focusing gels were run using the Ampholine PAGplate system developed by Pharmacia Biotech (Sweden) in the pH range of 3-9. pl was also determined using the MinpHor system developed by Rainin Co. (USA) with the broad range ampholyte mixture pH 3-9. The isoelectric point of the protein was determined to be approximately 5.6.

2.7 Determination of Molecular Weight

Molecular weight was determined by gel permeation chromatography and gel electrophoresis. SDS-PAGE gels (gradient 8-25%) were run according to the method of Laemmli (Laemmli, 1970, Nature 227, 680-685). Proteins were stained with Coomassie brilliant blue. Gel permeation chromatography was performed by HPLC on a 75×300 mm TosoHaas TSK-GEL GS3000SW XL column with a mobile phase of 200 mM potassium phosphate pH 6.8, 150 mM sodium chloride. Bio-Rad gel filtration standard mixture (MW 670,000-1,350) was used as the marker. The flow rate was 1.0 ml/min and the eluate was monitored at 280 nm. Fractions were collected and assayed for esterase activity. A single band at 80,000 Dalton was observed by SDS-PAGE; gel filtration chromatography of the enzyme indicated that the enzyme is a monomer in the native state.

2.8 Determination of Carbohydrate Content of Enzyme

Removal of carbohydrate with recombinant peptide N-glycosidase was performed as described by Elder et al., Proc. Natl. Acad. Sci. USA, 79, 4540-4544 (1982), and endoglycosidase H as performed by Trimble et al., Anal. Biochem. 141, 515-522 (1984). Native and deglycosylated enzymes were then analyzed by SDS-PAGE as described in Example 2.7 to determine carbohydrate loss. Treatment of the enzyme with endoglycosidases resulted in a 15-20% reduction of molecular weight to approximately 62,000 Dalton.

2.9 Determination of N-Terminal Amino Acid Sequence

The amino-terminal sequence was determined by automated Edman degradation at the Cornell University Biotechnology Analytical Facility. The amino terminal sequence obtained from the purified enzyme was H₂N-Thr-Asn-Pro-Asn-Glu-Pro-Pro-Pro-Val-Val-Asp-Leu-Gly-Tyr-Ala (SEQ.ID.NO.:5).

### EXAMPLE 3

### Cloning of Cephalosporin Esterase Gene from Rhodosporidium toruloides

3.1 Preparation of Chromosomal DNA of R. toruloides

Seed media culture was inoculated at 4% with a frozen culture of Rhodosporidium toruloides (ATCC 10657). The culture was grown at 28° C. for 24 hours in 2% glucose, 1% yeast extract, 1% Bacto-peptone, 0.5% KH₂PO₄, pH 6.0. Cells were harvested by centrifugation and washed once in buffer containing: 1 M sorbitol, 50 mM sodium citrate pH 5.4. Cells were centrifuged again and resuspended in wash buffer containing 0.5% lysing enzymes (Sigma Chemical Co., USA) at 37° C. for 3 hours. Spheroplasts were collected by centrifugation and digested in 100 mM NaCl, 10 mM Tris-HCl pH 8.0, 25 mM EDTA, 1.0% SDS and 100 μg/ml proteinase K. The solution was incubated at 50° C. for 16 hours. The mixture was extracted twice, first with phenol:chloroform:isoamyl alcohol (25:24:1), then with chloroform:isoamyl alcohol (24:1) and the DNA was precipitated with ethanol (70%). The DNA was recovered by centrifugation and washed with 70% ethanol. The DNA pellet was dissolved in TE (10 mM Tris-HCl pH 8.0, 1 mM EDTA) and 100 μg/ml RNase A and incubated at 37° C. for 16 hours. The organic extractions and ethanol precipitation were repeated and the DNA was dissolved in TE. The DNA concentration was determined spectrophotometrically.

3.2 Construction of Genomic DNA Library of R. toruloides

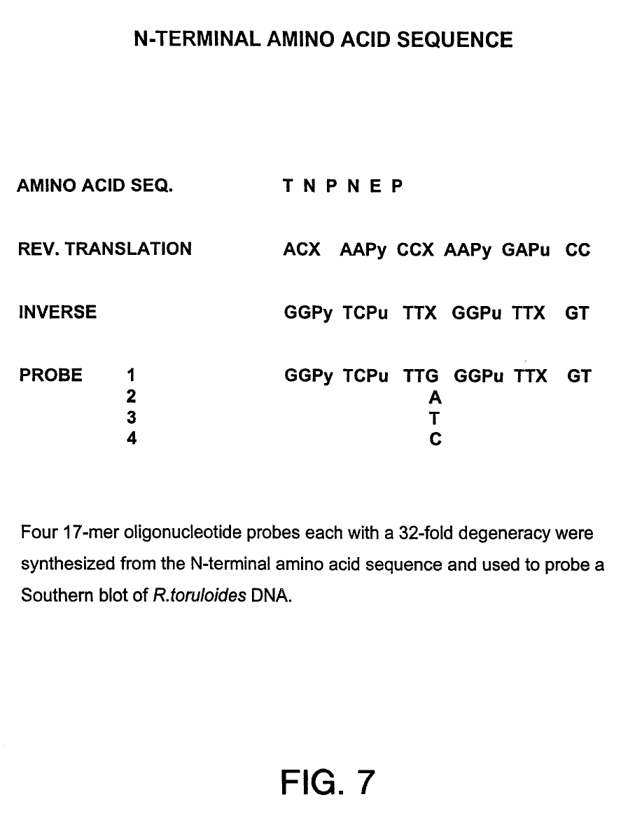

From the N-terminal amino acid sequence (Example 2.9) four 17-mer oligonucleotide probes were synthesized (FIG. 7), end-labeled with [γ-³²P]ATP, and used to probe a southern blot of R. toruloides chromosomal DNA digested with restriction endonucleases BamHI and PstI. Hybridization was conducted in TMAC (tetramethylammoniumchloride, Sigma Chemical, USA) buffer at 46.8° C. for 48 hours. A 3 kb BamHI fragment hybridized to one of the probes. The 3 kb BamHI fragment was isolated and ligated to pBluescript II KS+phagemid (Stratagene, USA) cleaved with BamHI and treated with bacterial alkaline phosphatase. The ligation mixture was used to transform E. coli XL1-blue cells [E. coli, recA1 endA1 gyrA96 thi-1 hsdR17 supE44 relA1 lac(F′ proAB lacI^{q}ZΔM15 Tn10)] by electroporation at 2.5 Kvolts, 200 ohms, 25 μFd. The transformants were selected on LB agar (1% tryptone, 0.5% yeast extract, 0.5% NaCl, 1.5% agar) containing 100 μg/ml ampicillin.

3.3 Selection of Clone Containing Cephalosporin Esterase Gene

Colony blots of the genomic library were prepared and screened with the N-terminal oligonucleotide probe. Twelve clones were initially selected for further evaluation. Plasmid DNA was isolated from each transformant using the TELT mini-prep method (He et al., 1990, Nucl. Acids Res. 18,1660). Southern analysis of these clones identified two that hybridized to the probe. Translation of the adjacent DNA sequence produced an amino acid sequence that was identical to the N-terminal protein sequence. Further analysis of the 3 kb BamHI fragment by primer extension and Southern blotting determined the location and orientation of the esterase gene within the fragment.

3.4 cDNA Cloning

A cDNA clone was produced by 3′RACE (rapid amplification of cDNA ends, Life Technologies, USA). Total RNA from R. toruloides was isolated using Trizol reagent (Life Technologies, USA) and further purified by lithium chloride precipitation. First strand cDNA was prepared by reverse transcription from an adapter primer. The RNA template was digested with RNase H and the cDNA was amplified by PCR using a gene-specific primer and an adapter primer. The coding region was amplified and mutagenized by a second round of PCR using an internal gene-specific primer which included the putative translation start site and an NcoI restriction site at the translation start site for subsequent cloning into expression vectors. This produced a 1.9 kb fragment which was gel purified. Restriction analysis and nucleotide sequencing of this fragment confirmed that it contained the esterase gene. To further facilitate cloning into an expression vector, another cDNA clone was developed that included a BspHI site at the beginning of the mature peptide and a BamHI site at the 3-end of the gene.

3.5 Determination of Nucleotide Sequence

The nucleotide sequence was determined by the dideoxy chain termination method (Sanger et al., 1977, Proc. Natl. Acad. Sci. USA 74, 5463-5467) using the Taq Track fmol DNA sequencing systems (Promega, USA). T3, T7, and synthesized internal primers were used to sequence the entire gene from both strands. Electrophoresis was performed on a 7% Long Ranger (AT Biochem., USA) polyacrylamide gel containing 7M urea in TBE buffer (89 mM Tris, 89 mM boric acid, 1 mM EDTA, pH 8.0) at 2700 volts. The complete nucleotide sequence is shown in FIG. 8. The coding cDNA region is 1716 bp long and codes for a 572 amino acid protein of molecular weight 61.3 kD. This is consistent with the deglycosylated form of the enzyme (Example 2.8). The N-terminal protein sequence determined from the DNA sequence is identical to the protein sequence identified in Example 2.9. This sequence begins 28 residues down from the putative ATG translation start site. The cDNA clone was also sequenced for comparison to the genomic clone. The gene contains five introns which are identified in FIGS. 9A and 9B.

### EXAMPLE 4

### Construction of Fungal Vectors for Esterase Expression

4.1 Reconstruction of the Esterase Gene Plasmid

### A. Subcloning of Genomic Esterase Coding Region

Five μg of the R. toruloides genomic esterase gene plasmid, pBMesterase1, was cleaved with PstI and BamHI and ligated to the vector pBluescript II KS+(Stratagene, USA) at the PstI and BamHI sites. The ligation mixture was transformed to DH5α MCR competent cells [E. coli F⁻ mcrA Δ(mrr-hsdRMS-mcrBC)φ80dlacZΔM15Δ(lacZYA-argF)U169 deoR recA1 endA1 phoA supE44λ⁻thi-1 gyrA96 relA1] and plated on LB agar containing ampicillin at 100 μg/ml. Ampicillin resistant colonies were screened for inserts with a PstI/BamHI cleavage. The constructed plasmid was designated as pBMesterase1a (FIG. 2).

An oligonucleotide fragment (5′-GATCACCCGGGT-3′) 3′-TGGGCCCACTAG-5′ (SEQ.I.D.NO.:6) was synthesized to convert the BamHI site of pBMesterase1a to a SmaI site. The oligonucleotide fragment was kinased and ligated to pBMesterase1a which had been cleaved with BamHI and treated with bacterial alkaline phosphatase. The ligation mixture was transformed and colony minipreps were checked for the replacement of the BamHI site with a SmaI site. A confirmed plasmid was designated as pBMesterase1b (FIG. 2).

### B. Cloning of the R. toruloides Esterase Gene Promoter

a. Cloning of the esterase gene promoter: A DNA fragment from the 5′ end of the esterase gene was digoxigenin labeled and used to probe a Southern blot of R. toruloides chromosomal DNA cleaved with PstI. An 1.6 kb PstI fragment was determined to include the esterase promoter region and the region encoding the N-terminal portion of the esterase protein. The 1.6 kb PstI genomic DNA fragments were isolated and ligated to pBluescript II KS+ vector cleaved with PstI and treated with bacterial alkaline phosphatase. The ligation mixture was used to transform DH5α MCR competent cells. The transformants were selected on LB agar containing 100 μg/ml ampicillin.

b. Colony blot: After plating transformation mixture on selective plates and overnight growth, plates were placed at 4° C. for 2 hours. A MagnaGraph 0.45 μm nylon filter (Micron Separations, USA) was placed on each plate for 2 minutes. Filters were gently removed from the plates and dried with colony side up for 10 minutes. Filters were placed colony side up on Whatman 12.5 cm filter paper disks saturated with 2 ml of 10% SDS for 10 minutes, 0.5 M NaOH, 1.5 M NaCl for 10 minutes, 1 M Tris-HCl pH 8.0, 1.5 M NaCl for 5 minutes and 2×SSC (0.3M NaCl, 20 mM sodium citrate, pH 7.0) for 15 minutes. Filters were crosslinked with UV irradiation using a GS Gene Linker UV Chamber (Bio-Rad Laboratories, USA) at a dosage of 150 mJoule. Filters were treated with 3×SSC, 0.1% SDS at 65° C. for 60 minutes, then rubbed gently with gloves to remove cell debris. Filters were then washed in 2×SSC for 5 minutes and allowed to air dry.

c. Colony hybridization: Filters were prehybridized at 39° C. for 30 minutes in 3 ml of Dig-Easy Hyb Buffer (Boehringer Mannheim catalog #160358, Boehringer Mannheim Corporation, USA) for each filter. A glass petri dish was used to house the filters with a glass mesh placed between each filter and agitated at 100 rpm. Fifty μl of a 1.6 kb digoxigenin labeled esterase specific probe was diluted in 1 ml of Dig-Easy Hyb Buffer and denatured in a boiling water bath for 10 minutes, then immediately placed on ice for 2 minutes. The prehybridization solution was poured off the filters to a 50 ml tube (Falcon#2098, Beckton Dickinson Labware, USA). The probe was added to this solution and pipetted back onto the filters. The filters were then hybridized at 39° C., 50 rpm overnight. The filters were washed two times in 2×SSC, 0.1% SDS for 5 minutes at 25° C. and then washed two times in 0.1×SSC, 0.1% SDS for 15 minutes at 65° C. The filters were incubated 3 hours at 25° C. with 100 ml of Genius Buffer 1 (100 mM Tris-HCl,100 mM NaCl, pH 7.5) with 1% blocking reagent (Boehringer Mannheim catalog #1096176). Anti-digoxigenin Fab (Boehringer Mannheim catalog #109327) was diluted 1:15,000 in 100 ml of Buffer 1 with 1% blocking reagent. Blocking solution was removed from the filters and replaced with the antibody solution. Filters were incubated in the antibody solution 30 minutes at 25° C. with gentle agitation. The antibody solution was removed and the filters were washed two times, 15 minutes each, with 100 ml of Genius Buffer 1 with 0.3% Tween 20. After the final wash, the filters were incubated 2 minutes in Genius Buffer 3 (100 mM Tris-HCl, pH 9.5, 100 mM NaCl, 50 mM MgCl₂). The excess solution was removed from the filters and 0.5 ml of CSPD (C₁₈H₂₀ClO₇PNa₂ Boehringer Mannheim catalog #1655884) diluted 1:100 in Genius Buffer 3 was applied to each filter and spread to cover the entire surface. The filters were placed within a plastic sheet protector and incubated 5 minutes at 25° C. The excess solution was blotted from the filter surface and the filters were transferred to a fresh plastic sheet protector. The filters were incubated at 37° C. for 15 minutes and placed on X-ray film (X-Omat AR, Eastman Kodak Company, USA) for 1 hour. Upon development of the film a single hybridization signal was observed.

The corresponding colony was picked to 1 ml LB (1% tryptone, 0.5% yeast extract, 0.5% NaCl) with 30 μg/ml ampicillin for a plasmid miniprep. This plasmid with the 1.6 kb PstI insert was designated as pBMesterase2 (FIG. 3).

### C. Reconstruction of the Esterase Coding Sequence and Promoter

The esterase promoter vector, pBMesterase2, was cleaved with PstI and separated on a 0.7% agarose gel to isolate the 1.6 kb promoter fragment. This fragment was then ligated to the PstI cleaved and bacterial alkaline phosphatase treated pBMesterase1b. The ligation mixture was transformed to DH5α MCR competent cells and plated on LB agar with 100 μg/ml ampicillin. The transformants were analyzed for the presence of inserts. An EcoRI/BamHI digest was used to determine the insert orientation. From this vector, pBMesterase3, an EcoRV/SmaI digest removes an intact esterase coding sequence and about 750 bp of the promoter region on a 3.8 kb fragment (FIG. 3).

4.2 Construction of Fungal Vector pSJC62.3

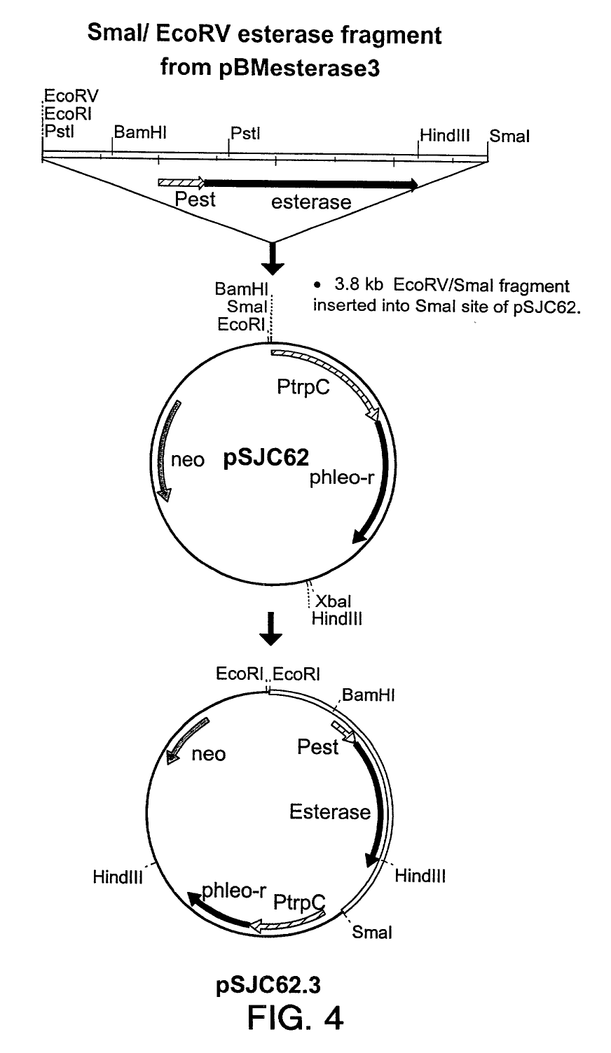

A 3.8 kb SmaI/EcoRV fragment from vector pBMesterase3 containing the genomic esterase gene and the R. toruloides promoter was gel purified and ligated to a fungal transformation vector pSJC62 (U.S. Pat. No. 5,516,679), which had been cleaved with BamHI, the 5′ protruding ends were filled in with Klenow enzyme (E. coli DNA polymerase I large fragment) and dephosphorylated with bacterial alkaline phosphatase. DH5α MCR competent cells (Life Technologies, USA) were transformed with the ligation mixture and plated to a LB agar plate containing 30 μg/ml of neomycin. Plasmid minipreps were performed on 12 colonies and 6 were found to contain inserts. An EcoRI digest was performed to determine the orientation of the insert. Plasmid pSJC62.3 contains the esterase gene transcribed in the same orientation of the phleomycin resistance gene (FIG. 4). A large scale plasmid preparation was performed on the DH5α MCR(pSJC62.3)⁺ culture to yield a sufficient quantity of plasmid for fungal transformation.

4.3 Construction of Fungal Vector pBMesterase11

Vectors containing the R. toruloides esterase gene under the control of the A. nidulans trpC promoter were constructed to achieve higher expression levels of the esterase in A. chrysogenum.

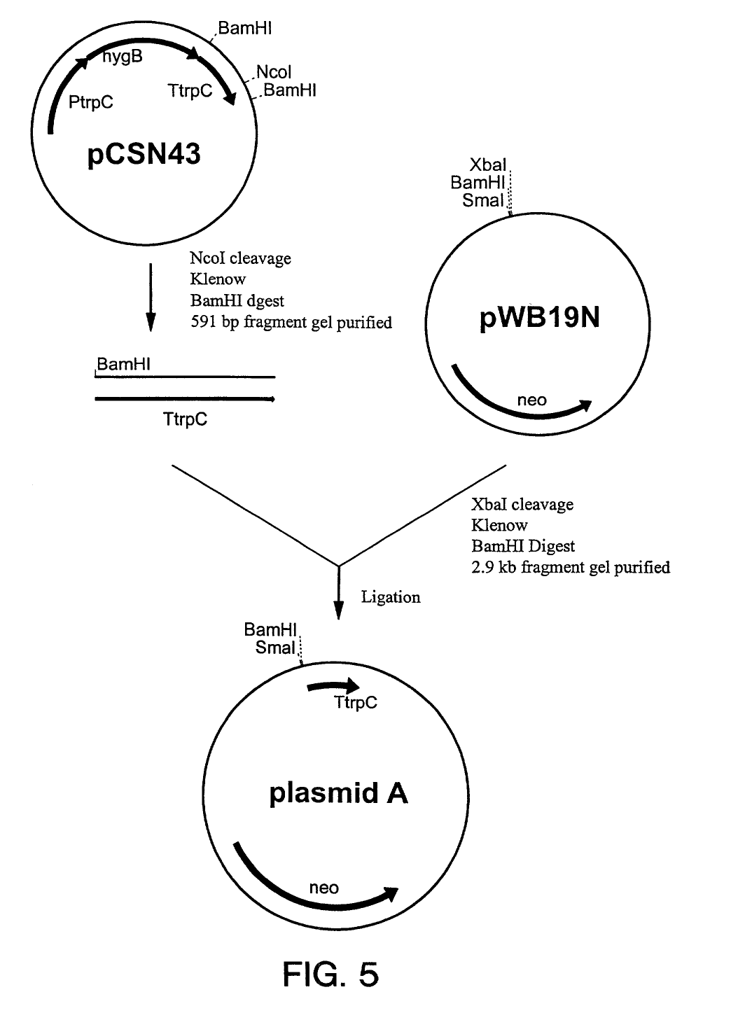

Plasmid pCSN43 (Staben et al., 1989, Fungal Genet. Lett. 36, 79-81) was digested with NcoI, filled in with Klenow enzyme and then cleaved with BamHI. The digest was separated on a 0.7% agarose gel. A 591 bp fragment containing the A nidulans trpC terminator with the 3′ terminal 153 bp of the trpC coding sequence was purified and ligated to plasmid pWB19N (U.S. Pat. No. 5,516,679) that had been cleaved with XbaI, filled in with Klenow enzyme and subsequently digested with BamHI. The ligation reaction was transformed to DH5α MCR competent cells and the resultant plasmid was designated as plasmid A (FIG. 5).

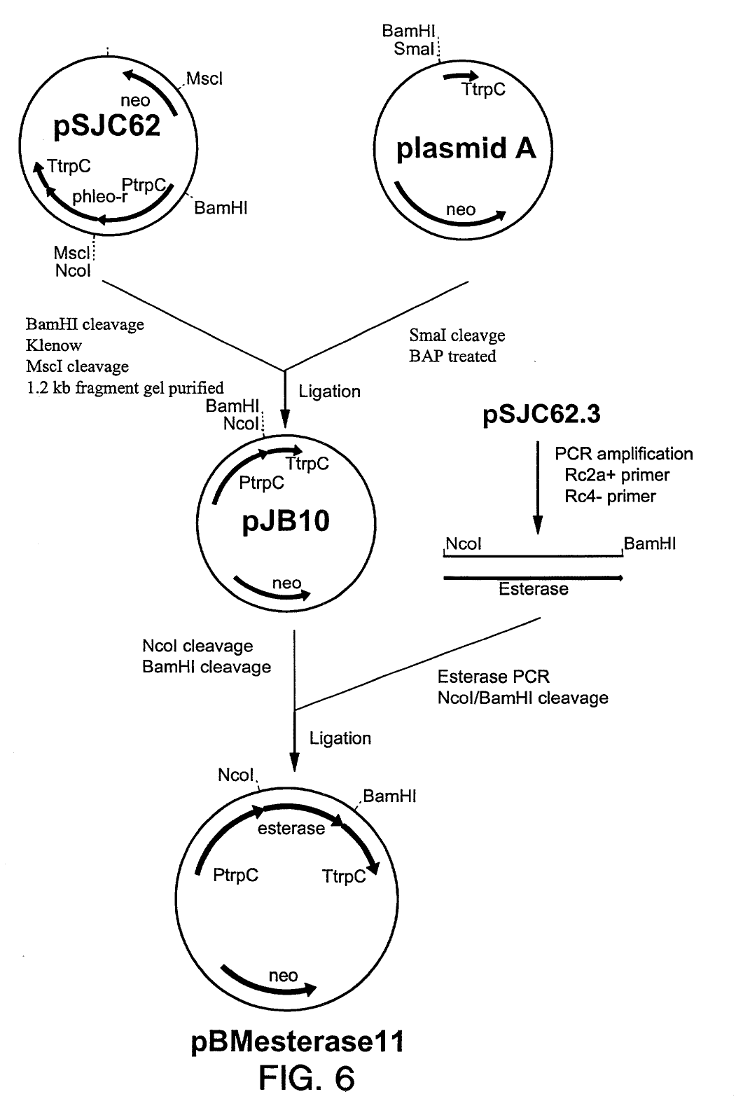

Plasmid pSJC62 was cleaved with BamHI, filled in with Klenow enzyme and then digested with Mscl. An 1.2 kb fragment containing the trpC promoter and the NcoI site present at the start codon of the Streptoalloteichus hindustanus phleomycin resistance gene was purified on a 0.7% agarose gel. This fragment was ligated to plasmid A which had been cleaved with SmaI and treated with bacterial alkaline phosphatase. This ligation mixture was transformed to DH5α MCR competent cells. The resultant plasmid was named pJB10 (FIG. 6).

In order to generate NcoI and BamHI cleavage sites for the insertion of the genomic esterase gene into plasmid pJB10, two oligonucleotide primers were synthesized for PCR reaction; primer Rc2a⁺(5′-ACTCGCCGCCATGGTCCTTAACCTCTTCAC-3′(SEQ. I.D.NO.:7, corresponding to N.T. #67 to N.T. #96 in SEQ.I.D.NO.:3) with a C→G change at N.T. #80 to generate a NcoI site; another primer Rc4⁻(5′-GAAAGACCCCTAGAGACCCGCGTTCACCGA-3′(SEQ.I.D.NO.:8, corresponding to N.T. #2117 to N.T. #2088 in SEQ.I.D.NO.:3) with a G→C change at N.T. #2110 to generate a BamHI site. The genomic esterase gene fragment including the leader sequence was amplified by PCR from vector pSJC62.3. The PCR reaction mixture consisted of: 1 μl of 1 ng/μl pSJC62.3, 4 μl 2.5 mM dNTPs, 2 μl of 10 μM primer Rc2A⁺, 2 μl of 10 μM primer Rc4, 5 μl of Pfu 10×buffer, 1 μl Pful enzyme (Stratagene, USA). The reaction conditions were 96° C. for 5 minutes, then 32 cycles of the following steps: 96° C. for 30 seconds, 68° C. for 30 seconds and 72° C. for 4 minutes. After 32 cycles the reaction was extended for 10 minutes at 72° C. The 2,048 bp esterase PCR product was digested with NcoI and BamHI and gel purified on a 0.7% agarose gel. The genomic esterase gene fragment was ligated to plasmid pJB10 that had been cleaved with NcoI and BamHI. The ligation reaction was transformed to DH5α MCR competent cells. The resultant plasmid was designated as pBMesterase11 (FIG. 6).

### EXAMPLE 5

### Acremonium chrysogenum Transformation

5.1 Transformation Method

One ml of a frozen vegetative stock culture of Acremonium chrysogenum strain BC1385 was used to inoculate 100 ml of PV media (2.4% malt extract, 2.7% yeast extract, 1% peptone) in a 500 ml Erlenmeyer flask (Basch et al., 1998, J. Ind. Microbiol. Biotechnol. 20, 344-353). The culture was incubated in a Model G25 shaker incubator (New Brunswick Scientific, USA) at 250 rpm for 64 hours at 28° C. The mycelia were harvested by vacuum filtration through a 30 μm mesh nylon filter (Spectra/Mesh Nylon N, Spectrum Medical Industries, USA) and washed with sterile deionized H₂O. The mycelia were then weighed and resuspended in filter sterilized Neutral Mcllvaine's buffer (0.1 M citric acid, 0.2 M Na₂HPO₄, pH 7.1) with 10 mM DTT in a ratio of 1 g mycelia per 20 ml buffer. The mixture was incubated in a Model G25 shaker incubator at 150 rpm for 90 minutes at 28° C. The mycelia were again harvested by filtration through a 30 μm mesh nylon filter and washed with sterile deionized H₂O. The mycelia were resuspended in filter sterilized Isotonic and Acidic Mcllvaine's buffer (0.1 M citric acid, 0.8 M NaCl, 20 mM MgSO₄, 0.2 M Na₂HPO₄, pH 6.35) containing Novozyme 234 (InterSpex Products, Inc., USA) at a concentration of 4 mg/ml. Ten ml of the above buffer was used for every gram of mycelia. The mixture was incubated at 28° C. in a Model G25 shaker incubator at 100 rpm for 60 minutes. The mycelia clumps were dissociated by pipetting up and down 10 times with a 10 ml glass pipette. Four volumes of washing buffer (0.8 M NaCl, 20 mM MgSO₄) was added and the entire solution was filtered through a sterilized glass funnel loosely packed with glass fiber. The filtrate was collected and centrifuged at 850×g for 8 minutes at room temperature in a Beckman TJ-6 centrifuge in a TH-4 swinging bucket rotor. The supernatant was decanted immediately. The protoplast pellet was washed twice in ½ volume of washing buffer at room temperature and centrifuged at 850×g for 8 minutes. The pellet was then resuspended in 0.8 M NaCl to a concentration of 3.5×10⁸ protoplast/ml. To 1 ml of protoplast, 5 μl dimethylsulfoxide and 80 μl of 1 M CaCl₂ solution was added. Twenty μg DNA in 20 μl TE and 4 μl of heparin (10 mg/ml) were added to a 14 ml polypropylene tube (Falcon #2059, 17×100 mm tube, Becton Dickinson Labware). For the transformation of plasmid pBMesterase11 which does not have a phleomycin resistance gene for the selection of fungal transformants, a mixture of 10 μg pBMesterase11 and 10 μg pSJC62 was used to introduce pBMesterase11 into host cells by cotransformation. One hundred μl of protoplasts were added to the DNA tube, followed by 20 μl of 40% polyethylene glycol-4000. The solution was mixed gently and incubated 10 minutes at room temperature. One ml of 40% polyethylene glycol-4000 was added, mixed gently, then 10 ml of molten (50° C.) top agar (0.8M NaCl, 0.7% agar) was added. Five ml of agar was pipetted to two plates pre-poured with 20 ml of regeneration agar (3% Trypticase Soy broth, 10.3% sucrose, 2% agar). The plates were incubated at 28° C. for 24 hours then overlayed with 5 ml of top agar containing phleomycin at a concentration of 12 μg/ml (the final concentration of phleomycin is 2 μg/ml). Transformants were observed after 2 weeks incubation at 28° C.

5.2 Verification of Transformants

### A. Isolation of A. chrysogenum Genomic DNA

One ml of a frozen vegetative stock culture from transformants DC1, DC2, DC3, DC11, and DC14 was inoculated in 30 ml of PV media. The mycelial cultures were grown, collected and treated with Novozyme 234 to form protoplasts as described above. The protoplast pellet was resuspended in 3 ml lysis buffer (0.7 M NaCl, 10 mM Tris-HCI, pH 8.0, 10 mM EDTA, 1% SDS) and incubated at 37° C. for 5 minutes. A volume of 0.3 ml of 10% cetyltrimethylammonium bromide (CTAB) in 0.7 M NaCl was added to the lysis mixture and incubated at 65° C. for 10 minutes. The solution was extracted twice with an equal volume of chloroform/isoamyl alcohol (24:1) to remove the CTAB-polysaccharide complex. The aqueous solution was collected and DNA was precipitated with the addition of 6 ml of 100% ethanol. The DNA pellet was collected by centrifugation, washed with 70% ethanol, dried 5 minutes under vacuum and resuspended in 500 μl of TE. Ten μl of RNase A (10 mg/ml) was added and incubated at 37° C. for 1 hour. Proteinase K solution was added to the tube to reach a final concentration of 400 μg/ml and incubated at 50° C. for 30 minutes. Sixty μl of 3 M NaCl was added and the mixture was extracted with an equal volume of phenol/chloroform/isoamyl alcohol mixture (25:24:1). The DNA was precipitated with two volumes of 100% ethanol for 1 hour at room temperature. The DNA was then pelleted by centrifugation, washed with 70% ethanol, dried, and resuspended in 400 μl of TE.

### B. Gel Electrophoresis and Blot

Five μg of genomic DNA was digested with 40 units of EcoRI in a total volume of 200 μl. The reaction was incubated for 3 hours at 37° C. Twenty μl of 3 M NaOAc pH 5.2 was added and mixed. The digests were then extracted once with an equal volume of phenol/chloroform/isoamyl alcohol (25:24:1). Two volumes of 100% ethanol were added and the DNA was precipitated for 30 minutes at −70° C. The DNA was pelleted by centrifugation for 10 minutes at 4° C., dried 5 minutes under vacuum and resuspended in 20 μl of TE. Eight μl of the digests were loaded on a 0.7% agarose gel in TAE buffer (40 mM Tris-acetate, pH 8.0, 1 mM EDTA) and separated at 12 V for 16 hours. The DNA was de-purinated for 10 minutes with 0.25 N HCl then rinsed with deionized H₂O. The DNA was transferred to a nylon membrane (Boehringer Mannheim catalog #1209 299) with 0.4 N NaOH using a Bio-Rad Model 785 Vacuum Blotter (Bio-Rad catalog #165-5000). After transfer, the DNA was crosslinked to the membrane by UV irradiation using a GS Gene Linker UV Chamber (Bio-Rad Laboratories) at a dosage of 125 mjoule.

### C. Hybridization

The filter was prehybridized at 50° C. for 30 minutes in 10 ml of Dig-Easy Hyb Buffer. Five μl of a PCR generated digoxigenin labeled probe (10 ng/μl) specific to the neomycin resistance gene was diluted in 1 ml of Dig-Easy Hyb Buffer and denatured in a boiling water bath for 10 minutes. The denatured probe was then placed on ice for two minutes. Five ml of the prehybridization solution was poured off the filters to a 14 ml polypropylene tube. The probe was added to this solution and pipetted back onto the filter. The filter was then hybridized at 50° C. overnight. The filter was washed two times in 2×SSC, 0.1% SDS 5 minutes at 25° C. and then washed two times in 0.5×SSC, 0.1% SDS 15 minutes at 65° C. The filter was then treated for the detection of Dig-labeled DNA hybrid as described in Example 4.1.B section c.

5.3 Status of Transforming Plasmids in Transformants

Genomic DNA was isolated from transformants DC1, DC2, DC3, DC11 and DC14 and from the host culture BC1385. The DNA was cleaved with EcoRI, separated on an agarose gel and transferred to a nylon membrane. The membrane was hybridized to a PCR generated probe specific to the neomycin resistance gene or the R. toruloides esterase gene. The developed Southern blot indicates that the gene probe hybridizes to the transformant DNA, but not to that of the untransformed host DNA. All transformed plasmid DNA also integrated into host chromosomes. Some of the transformants have multiple copies of the plasmid integrated.

### EXAMPLE 6

### Production of Desacetylcephalosporin C

6.1 Shake Flask Evaluation of Desacetylcephalosporin C Production

After two weeks incubation of transformation plates, phleomycin resistant transformants were transferred by sterile toothpicks to YE agar (1% malt extract, 0.4% yeast extract, 0.4% glucose, 2% agar, pH 7.3) plates and incubated for 7 days at 28° C. Colonies were then used to inoculate slants containing 6 ml of YE agar and grown 7 days at 28° C. Two ml of sterile deionized H₂O was used to resuspend the culture from each slant, 1 ml of the resuspended culture was then inoculated to 25 ml of seed media in a 125 ml Erlenmeyer flask. The seed cultures were cultivated in a shaker at 28° C. for 48 hours, 250 rpm. Two ml of the seed culture was then transferred to 20 ml of fermentation media in a 125 ml bi-indented Erlenmeyer flask, grown 7 days at 24° C., 250 rpm. Whole broth was used for an HPLC assay of the concentration of cephalosporin C and desacetylcephalosporin C. Fermentation controls were A. chrysogenum BC1385 culture with the addition of 100 μl of decanol-treated R. toruloides cells.

The results of shake flask evaluation of transformants revealed that all five transformants, three pSJC62.3 transformants and two pBMesterase11 transformants, were found to produce only desacetylcephalosporin C under standard shake flask screening conditions. An untransformed BC1385 culture with the addition of 100 μl of the decanol-treated R. toruloides cells was included as a control. The cephalosporin C and desacetylcephalosporin C titers of each strain is demonstrated in Table 3.

TABLE 3
Shake Flask Evaluation of the Recombinant A. chrysogenum Strains

| | | | Desacetyl- |
|---|---|---|---|
| | | Cephalosporin | cephalosporin |
| Strains | Vector | C (Units/gm*) | C (Units/gm*) |
| DC1 | pSJC62.3 | <0.1 | 87 |
| DC2 | pSJC62.3 | <0.1 | 98 |
| DC3 | pSJC62.3 | <0.1 | 96 |
| DC11 | pBMesterase11 | <0.1 | 99 |
| DC14 | pBMesterase11 | <0.1 | 91 |
| BC1385 + | — | <0.1 | 100 |
| R. toruloides | | | |

*relative units of cephalosporin C or desacetylcephalosporin C per gram of fermentation broth.

6.2 Esterase Gene Expression in A. chrysogenum

The R. toruloides cephalosporin esterase is a heavily glycosylated membrane associated protein, with the carbohydrate residues required for the protein's enzymatic activity. The nucleotide sequence of this gene indicates that there is a 28 amino acid leader sequence which is removed in the mature form of the cephalosporin esterase protein. Efforts to express this gene in E. coli have failed to produce detectable enzymatic activity. The heterologous expression of an active cephalosporin esterase enzyme in a A. chrysogenum host from the genomic gene with its endogenous Rhodosporidium promoter (e.g., gene construct in pSJC62.3) or with an Aspergillus trpC promoter (e.g., gene construct in pBMesterase11) indicates that: 1) the promoter must be recognized by the A. chrysogenum RNA polymerase; 2) the five introns are correctly spliced; 3) the leader sequence is removed; and 4) the protein must be glycosylated. In fact, most of the esterase gene transformants produce predominately desacetylcephalosporin C. As the transformation is performed with supercoiled DNA, some transformants are not desacetylcephalosporin C producers and probably have the cephalosporin esterase gene disrupted at integration.

What is claimed is:

1. A process for the direct production of desacetylcephalosporin C comprising culturing a strain of Acremonium chrysogenum containing nucleic acid encoding enzymes for cephalosporin C biosynthesis and a recombinant nucleic acid encoding Rhodosporidium cephalosporin esterase under conditions resulting in the synthesis of cephalosporin C and expression of cephalosporin esterase wherein the cephalosporin C so produced is converted to desacetylcephalosporin C.

2. The process of claim 1 wherein the chemical breakdown of cephalosporin C to 2-(D-4-amino-4-carboxybutyl)-thiazole-4-carboxylic acid is less than 40%.

3. The process of claim 1 wherein the chemical breakdown of cephalosporin C to 2-(D-4-amino-4-carboxybutyl)-thiazole-4-carboxylic acid is less than 30%.

4. The process of claim 1 wherein the chemical breakdown of cephalosporin C to 2-(D-4-amino-4-carboxybutyl)-thiazole-4-carboxylic acid is less than 20%.

5. The process of claim 1 wherein the chemical breakdown of cephalosporin C to 2-(D-4-amino-4-carboxybutyl)-thiazole-4-carboxylic acid is less than 10%.

6. The process of claim 1 wherein the chemical breakdown of cephalosporin C to 2-(D-4-amino-4-carboxybutyl)-thiazole-4-carboxylic acid is less than 5%.

7. The method of claim 1 carried out at a temperature of about 22° C. to about 29° C. and a pH of about 5.5 to about 7.5.

8. The method of claim 1 carried out at a temperature of about 25° C. to about 29° C. and a pH of about 6.2 to about 7.0, during the vegetative cell growth phase; at a temperature of about 22° C. to about 26° C. and a pH of about 5.7 to about 6.5 during the desacetylcephalosporin C production phase.

9. The method of claim 1 wherein the recombinant nucleic acid encoding Rhodosporidium cephalosporin esterase is DNA.

10. The method of claim 1 wherein the recombinant nucleic acid encoding Rhodosporidium cephalosporin esterase is DNA and part of a plasmid.

11. The method of claim 10 wherein the recombinant nucleic acid encoding Rhodosporidium cephalosporin esterase has the sequence of SEQ.ID.NO.:1 or 3.

12. The method of claim 10 wherein the plasmid is pSJC62.3.

13. The method of claim 10 wherein the plasmid is pBMesterase11.
